(19) United States
(12) Patent Application Publication
(10) Pub. No.: US 2025/0339709 A1
(43) Pub. Date: Nov. 6, 2025

(54) ß-RAY THERAPY APPARATUS AND USE METHOD THEREOF

(71) Applicant: PEKING UNION MEDICAL COLLEGE HOSPITAL, CHINESE ACADEMY OF MEDICAL SCIENCES, Beijing (CN)

(72) Inventors: Zhaohui ZHU, Beijing (CN); Jiarou WANG, Beijing (CN); Linlin LI, Beijing (CN); Rongxi WANG, Beijing (CN)

(73) Assignee: PEKING UNION MEDICAL COLLEGE HOSPITAL, CHINESE ACADEMY OF MEDICAL SCIENCES, Beijing (CN)

(21) Appl. No.: 18/865,933

(22) PCT Filed: Mar. 7, 2024
(86) PCT No.: PCT/CN2024/080421

§ 371 Date: Nov. 14, 2024

(30) Foreign Application Priority Data
Oct. 31, 2023 (CN) 202311434045.5

(51) Int. Cl. A61N 5/10

(52) U.S. Cl. CPC A61N 5/1028; A61N 5/1042; A61N 2005/1019; A61N 2005/1074; A61N 2005/1085; A61N 2005/1094

(57) ABSTRACT

Provided are a β-ray therapy apparatus and a method of using the same. The β-ray therapy apparatus includes a therapy apparatus body and a conformable assembly. The therapy apparatus body includes a β-ray source and a β-ray shielding assembly. The β-ray shielding assembly is provided with a mounting chamber and a window communicating with the mounting chamber, and the β-ray source is mounted in the mounting chamber. The conformable assembly includes a conformable frame and multiple conformable sheets, a transmission area is formed in the middle of the conformable frame, the conformable sheets are arranged on the conformable frame, and can move towards or away from the transmission area. A conformable area surrounding a site to be treated is formed in the transmission area by adjusting positions of the conformable sheets. The β-ray source can emit β-rays toward the conformable area through the window.

### CROSS-REFERENCE TO RELATED APPLICATION

This application is a Continuation-in-Part of International Patent Application No. PCT/CN2024/080421, filed on Mar. 7, 2024, which designates the United States, and which International Application, in turn, claims priority to Chinese Patent Application No. 202311434045.5, filed on Oct. 31, 2023, all of which are incorporated herein by reference in their entirety.

### TECHNICAL FIELD

The present disclosure relates to the field of external beam radiotherapy of nuclear medicine, and in particular to a β-ray therapy apparatus and a method of using the same.

### BACKGROUND

β-rays emitted by radionuclides can be used to effectively treat a variety of skin diseases, and especially have a good therapeutic effect in skin hemangioma and skin keloid. The β-ray applicator should be attached to the lesion surface during treatment.

At present, there are two types of β-ray applicators in common use: one type is a low-dose ³²P applicator, which can be taken home by patients after being attached, resulting in misalignment, falling off, lack of protection, loss, environmental pollution, and other problems. For example, an applicator based on radionuclide ³²P is provided in Chinese patent No. CN212416081 U, and includes a bearing body, and a supporting base capable of being buckled on the bearing body, and a bearing layer for bearing drugs is arranged at the top of the bearing body, and the bearing body can be attached to a diseased area. This scheme solves the problem that the existing ³²P applicator has no radiation protection function and low fitting degree. However, in order to achieve the above purpose, the sealing layer, the bearing layer, a substrate layer and the supporting base are all 3D (three-dimensionally) printed, which are printed after 3D scanning and data analysis of the lesion morphology. Therefore, this scheme needs to be made separately according to the difference of each lesion area, and does not have universal adjustability.

The other is a ⁹⁰Sr-⁹⁰Y applicator with fixed shape and dose, which cannot give different doses of β-ray therapy according to the shape, thickness, and state of the lesion. For example, a ⁹⁰strontium applicator is provided in Chinese application publication No. CN102049098 A, and has a handle, and a protective screen. The handle is provided with a groove in the middle, and an applicator at the lower end. The protective screen is provided with a retaining bead, and a spring. The protective screen is sleeved with the groove at the lower end of the handle, and the retaining bead can fix the protective screen. The purpose of this scheme is that the protective screen can be mounted and dismounted, which is convenient for operation and shortens the radiation exposure time, but in fact, the problem that the applicator cannot be adjusted in shape is not changed.

In conclusion, the existing applicator is not conformable, poor in conformability, or needs to be conformable by 3D printing, and the same apparatus cannot adapt to different lesion shapes, thicknesses, states and the like, which is not universal and adjustable.

### SUMMARY

An objective of the present disclosure is to provide a β-ray therapy apparatus and a method of using the same, so as to solve the problems in the prior art. By moving conformable sheets on a conformable frame to different positions, conformable areas with different shapes can be formed according to sites to be treated with different shapes, and then the sites to be treated can be irradiated by β-rays emitted by a β-ray source through the conformable areas. The same therapy apparatus can be used to match with the sites to be treated with different shapes, and has universality and adjustability.

To achieve the objective above, the present disclosure employs the following technical solution:

The present disclosure provides a β-ray therapy apparatus, including a therapy apparatus body, and a conformable assembly. The therapy apparatus body includes a β-ray source, and a β-ray shielding assembly. The β-ray shielding assembly is provided with a mounting chamber, and a window communicating with the mounting chamber. The β-ray source is mounted in the mounting chamber. The conformable assembly includes a conformable frame, and multiple conformable sheets, a transmission area is formed in the middle of the conformable frame, the conformable sheets are arranged on the conformable frame, and can move towards or away from the transmission area. A conformable area surrounding a site to be treated is formed in the transmission area by adjusting positions of the conformable sheets. The β-ray source emits β-rays towards the conformable area through the window.

In some embodiments, the conformable frame is an annular frame, a hollow region of the annular frame is the transmission area, multiple conformable sheets are divided into two groups, and the two groups of conformable sheets are slidingly arranged on two opposite sides of the annular frame, respectively.

In some embodiments, the periphery of the β-ray shielding assembly is provided with multiple first binding ring buckles, the periphery of the conformable frame is provided with multiple second binding ring buckles, and the first binding ring buckles and the second binding ring buckles are respectively configured for connecting with elastic straps.

In some embodiments, the β-ray therapy apparatus includes a β-ray irradiation window. The β-ray irradiation window is mounted at the window, and includes one or more blocking sheets. The blocking sheets move with respect to the window to change a size and shape of the window for adjusting the irradiation range of the β-rays emitted by the β-ray source.

In some embodiments, the blocking sheet is arranged on the β-ray shielding assembly in a penetrating manner, and moves towards or away from the window to shield or open the window.

In some embodiments, when multiple blocking sheets are provided, the multiple blocking sheets are divided into two groups, and the two groups of blocking sheets are slidingly arranged on two opposite sides of the β-ray shielding assembly, respectively.

In some embodiments, two groups of blocking sheets are arranged in one-to-one correspondence to form a plurality of pairs of blocking sheets. In each pair of blocking sheets, one is provided with a V-shaped protrusion, and the other is provided with a V-shaped groove in fit with the V-shaped protrusion. The V-shaped protrusion and the V-shaped groove extend in a width direction of the window.

In some embodiments, the β-ray therapy apparatus includes a controller. The controller is mounted on an outer side of the β-ray shielding assembly, and includes a display screen, an operation button, a dose measuring assembly, and an alerting or alarming assembly.

In some embodiments, the β-ray therapy apparatus includes an auxiliary device. The auxiliary device includes a supporting base, a host, and a mechanical arm. The supporting base is provided with a groove for placing the therapy apparatus body, the host is fixedly mounted on the mechanical arm, the host is provided with a closed storage area, and the mechanical arm is provided with a clamp for clamping the therapy apparatus body.

The present disclosure further provides a method of using the β-ray therapy apparatus above, including the following steps:
placing a conformable frame at a site to be treated, adjusting positions of conformable sheets according to a shape of the site to be treated, thus forming a conformable area surrounding the site to be treated;
applying ointment or gel to the site to be treated, and making an area applied with the ointment or gel flush with surfaces of the conformable sheets; and
buckling a therapy apparatus body on the conformable frame, and emitting β-rays by a β-ray source, where the β-rays irradiate to the area applied with the ointment or gel through the conformable area.

Compared with the prior art, the present disclosure has the following technical effects:

(1) By moving the conformable sheets on a conformable frame to different positions, the conformable areas with different shapes can be formed according to sites to be treated with different shapes, and then the sites to be treated can be irradiated by β-rays emitted by a β-ray source through the conformable areas. The same therapy apparatus can be used to match with the sites to be treated with different shapes, and has universality and adjustability.

(2) A β-ray irradiation window is provided, and the size and shape of the window can be changed by moving the blocking sheet, thus adjusting the irradiation range of the β-ray. Based on the existing conformable area, two-stage adjustment of the irradiation range of the β-ray can be formed, such that the β-ray can be controlled more accurately to irradiate a site to be treated, the amount of irradiation to a non-pathological location can be reduced, and normal tissues can be better protected.

(3) The periphery of the β-ray shielding assembly is provided with multiple first binding ring buckles, and the periphery of the conformable frame is provided with multiple second binding ring buckles. The first binding ring buckles and the second binding ring buckles are used to fix the therapy apparatus to a human body by the elastic straps, thus achieving twice fixations, increasing binding effect, fixing the therapy apparatus better, and preventing the influence on the irradiation effect caused by the displacement of the therapy apparatus.

(4) After the conformable area is formed by using the conformable sheets, the ointment or gel is applied to the site to be treated, and uneven lesion can be applied with the ointment or gel to the same level, such that the irradiation dose on the site with thick lesion (thin applying thickness) is more, while the irradiation dose on the site with thin lesion (thick applying thickness) is less, thus different degrees of lesions can be irradiated with different intensities to achieve the effect of intensity modulation.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a front cross-sectional view of a therapy apparatus body according to the present disclosure;

FIG. 2 is a top view of a controller according to the present disclosure;

FIG. 3 is a top cross-sectional view of a β-ray shielding assembly according to the present disclosure;

FIG. 4 is a top view of a blocking sheet according to the present disclosure;

FIG. 5 is a front view of a supporting base according to the present disclosure;

FIG. 6 is a top view of FIG. 5;

FIG. 7 is a front cross-sectional view of a conformable assembly according to the present disclosure;

FIG. 8 is a top view of FIG. 7;

FIG. 9 is a schematic diagram of the present disclosure including an auxiliary device therein;

FIG. 10 is a top view of FIG. 9.

In the drawings: 1—controller; 2—β-ray shielding assembly; 3—β-ray source; 4—blocking sheet; 5—supporting base; 6—conformable frame; 7—conformable sheet; 8—first binding ring buckle; 9—display screen; 10—operation button; 11—second binding ring buckle; 12—clamp; 13—mechanical arm; 14—host; 15—closed storage area.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide a β-ray therapy apparatus and a method of using the same, so as to solve the problems in the prior art. By moving conformable sheets on a conformable frame to different positions, conformable areas with different shapes can be formed according to sites to be treated with different shapes, and then the sites to be treated can be irradiated by β-rays emitted by a β-ray source through the conformable areas. The same therapy apparatus can be used to match with the sites to be treated with different shapes, and has universality and adjustability.

In order to make the objectives, features and advantages of the present disclosure more clearly, the present disclosure is further described in detail below with reference to the accompanying drawings and specific embodiments.

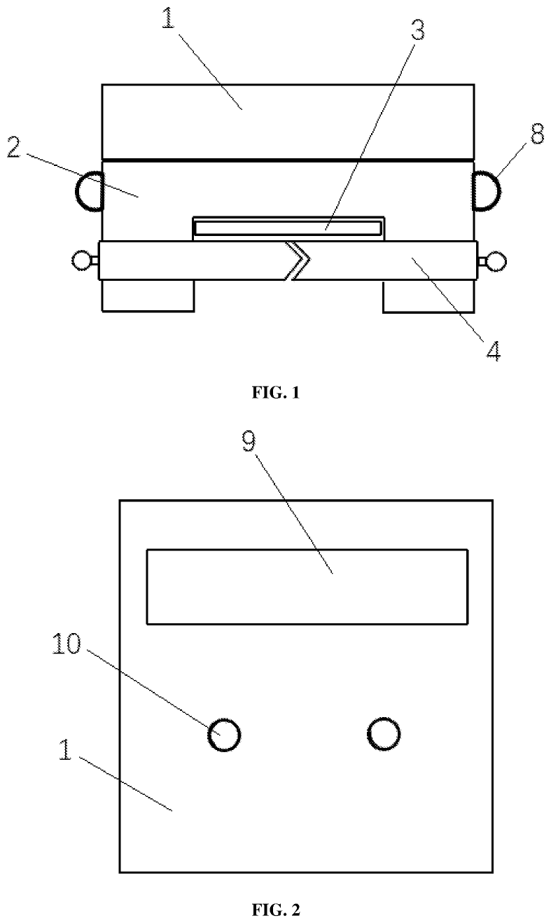

As shown in FIG. 1 to FIG. 8, the present disclosure provides a β-ray therapy apparatus, including a therapy apparatus body, and a conformable assembly. The therapy apparatus body is mainly used to provide a β-ray for irradiation, and the conformable assembly is used for limiting the irradiation range of the β-ray. Specifically, the therapy apparatus body includes a β-ray source 3, and a β-ray shielding assembly 2. The β-ray shielding assembly 2 is provided with a mounting chamber and a window communicating with the mounting chamber. The β-ray source 3 is mounted in the mounting chamber, which may be a ⁹⁰Sr-⁹⁰Y sealing source, ³²P or other radioactive sources capable of emitting β-ray nuclides. The β-ray shielding assembly 2 may be made of a lightweight material such as aluminum, resin, or plastic, and is used for shielding the β-ray source 3 from emitting rays to other directions except for a lesion direction, thus protecting the safety of the people around. The conformable assembly includes a conformable frame 6 and multiple conformable sheets 7. A transmission area for the β-ray to penetrate is formed in the middle of the conformable frame 6. The conformable sheets 7 are arranged on the conformable frame 6. An end, away from the conformable frame 6, of each conformable sheet 7 is provided with a handle, and the handle can be used to pull or push the conformable sheet 7 to move towards or away from the transmission area, thus adjusting the area and position for blocking the transmission area. When there are multiple conformable sheets 7, by adjusting the lengths of different conformable sheets 7 penetrating into the transmission area (that is, the blocking area and position are different), a conformable area surrounding a site to be treated can be formed in the transmission area by adjusting positions of the conformable sheets 7 according to the shape of the site to be treated, and the shape of the conformable area is basically identical with that of the site to be treated. It should be noted that the more the number and the smaller the size of the conformable sheets 7, the finer the conformable area can be. Here, the specification and size of the conformable sheet 7 are not restricted. In addition, there is no requirement on neither the shape of the β-ray shielding assembly 2 nor the shape of the window, which may be circular, rectangular or other shapes, and the opening range of the window can cover the area of the site to be treated, such that the adjusted conformable area can cover the site to be treated. The β-ray source 3 emits β-rays in the mounting chamber, such that the β-rays can emit β-rays towards the conformable area through the window, thus irradiating and treating the diseased site. In conclusion, by moving conformable sheets 7 on the conformable frame 6 to different positions, the conformable areas with different shapes can be formed according to sites to be treated with different shapes, and then the sites to be treated can be irradiated by β-rays emitted by the β-ray source 3 through the conformable areas. The same therapy apparatus can be used to match with the sites to be treated with different shapes, and has universality and adjustability.

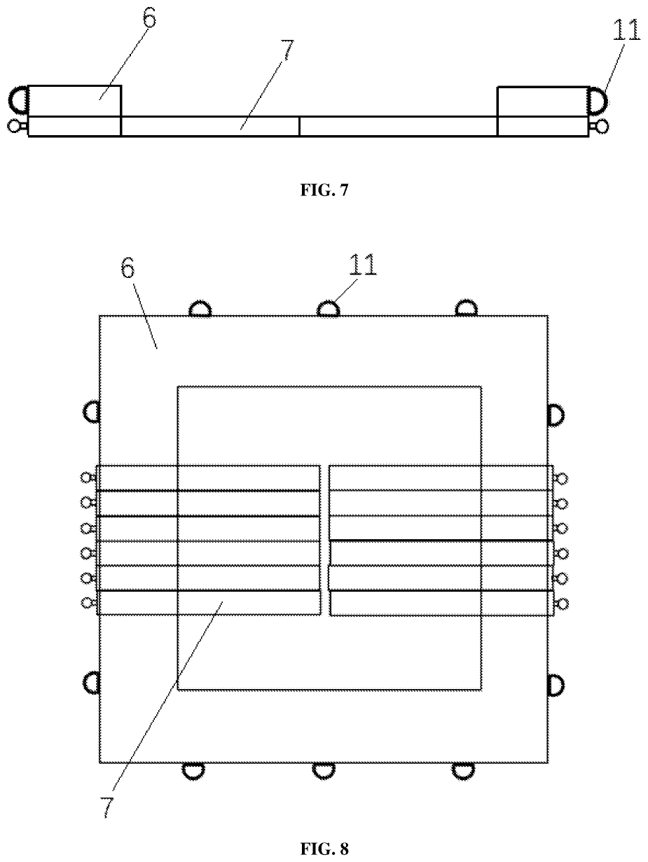

As shown in FIG. 7 and FIG. 8, the conformable frame 6 may be an annular frame, a main body of the annular frame is annular, and the annular shape here may be rectangular, circular or other shapes that can form a ring, not specifically a circular ring. A hollow region of the annular frame is a transmission area, multiple conformable sheets 7 are divided into two groups, and each group includes multiple conformable sheets 7 arranged in parallel. The two groups of conformable sheets 7 are slidingly arranged on two opposite sides of the annular frame, respectively, and the blocked width shape and area of the transmission area can be changed by relatively inserting or pulling out the conformable sheets 7. Further, the conformable frame 6 may be a rectangular frame shown in FIG. 8, while the conformable sheet 7 is a long-striped rectangular sheet, and two groups of rectangular sheets are located on two opposite edges of the rectangular frame, respectively.

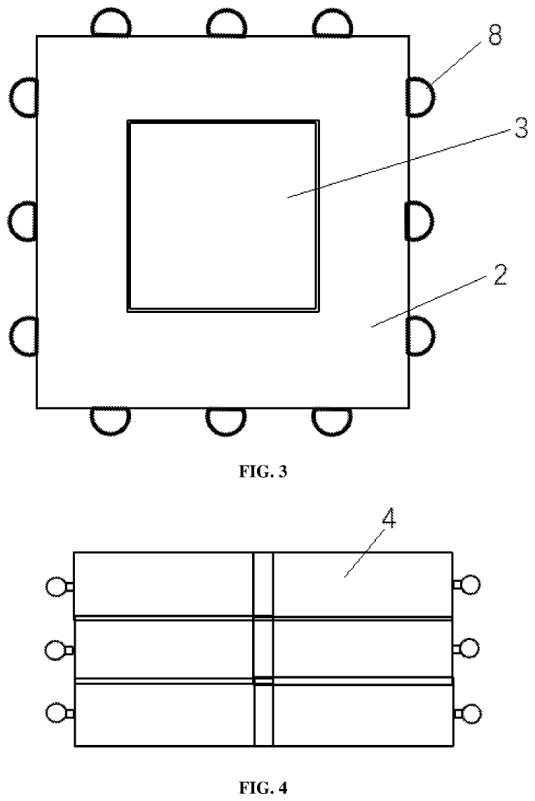

As shown in FIG. 1, FIG. 3, FIG. 7 and FIG. 8, the periphery of the β-ray shielding assembly 2 is provided with multiple first binding ring buckles 8, and the periphery of the conformable frame 6 is provided with multiple second binding ring buckles 11. The first binding ring buckles 8 and the second binding ring buckles 11 each are arranged at intervals, and at least two binding ring buckles are arranged on each edge. The first binding ring buckles 8 and the second binding ring buckles 11 are used to connect elastic straps, and the therapy apparatus can be fixed to the human body by the first binding ring buckles 8 and the second binding ring buckles 11 through the elastic straps, thus achieving twice fixations, increasing binding effect, fixing the therapy apparatus better, and preventing the influence on the irradiation effect caused by the displacement of the therapy apparatus.

As shown in FIG. 1 and FIG. 4, the β-ray therapy apparatus includes a β-ray irradiation window. The β-ray irradiation window is mounted at the window of the β-ray shielding assembly 2. The β-ray irradiation window includes one or more blocking sheets 4. The blocking sheet 4 is made of a lightweight material such as aluminum, resin, or plastic. The blocking sheet 4 may be directly arranged on the β-ray shielding assembly 2 in a penetrating manner, or arranged on a window structure, and then mounted on the window through the window structure. The blocking sheet 4 can move horizontally or longitudinally when moving with respect to the window, preferably in a longitudinal moving mode of inserting or pulling out. An end, away from the β-ray shielding assembly 2, of the blocking sheet 4 is provided with a handle, and the handle is used to drive the blocking sheet 4 to move. When the therapy apparatus body does not perform irradiation, the window can be completely closed by the blocking sheet 4 to avoid improper emission of β-rays. When only one blocking sheet 4 is provided, the blocking sheet 4 can be used to reciprocate in one direction to change the size of the window. When multiple blocking sheets 4 are provided, the window size can be changed by moving different blocking sheets 4, and the size of the window can be in fit with the shape of the site to be treated at the same time. The change of the window can adjust the irradiation range of the β-rays emitted by the β-ray source 3. More specifically, on the basis of forming the conformable area by using the existing conformable assembly, two-stage adjustment of the irradiation range of the β-ray can be formed, such that the β-ray can be controlled more accurately to irradiate a site to be treated, the amount of irradiation to a non-pathological location can be reduced, and normal tissues can be better protected.

More specifically, the β-ray shielding assembly 2 may be provided with a through hole or slot, the blocking sheet 4 may be arranged in the through hole or slot in a penetrating manner, and can move towards or away from the window to shield or open the window.

When multiple blocking sheets 4 are provided, the multiple blocking sheets 4 are divided into two groups, and each group include multiple blocking sheets 4 arranged in parallel. The two groups of blocking sheets 4 are slidingly arranged on two opposite sides of the β-ray shielding assembly 2, respectively. When the β-ray shielding assembly 2 is a rectangular frame, the blocking sheet 4 is a rectangular sheet, and the rectangular sheets are arranged on two opposite edges of the rectangular frame.

Two groups of blocking sheets 4 are arranged in one-to-one correspondence to form multiple pairs of blocking sheets. In each pair of blocking sheets 4, one blocking sheet is provided with a V-shaped protrusion, the other blocking sheet is provided with a V-shaped groove in fit with the V-shaped protrusion. The V-shaped protrusion and the V-shaped groove extend in a width direction of the window. When the blocking sheets 4 close to each other, the V-shaped protrusions can be inserted into the V-shaped grooves to form up-and-down shielding, thereby reducing a gap, and improving the blocking effect to the β-rays.

As shown in FIG. 1 and FIG. 2, the β-ray therapy apparatus includes a controller 1. The controller 1 is mounted on an outer side of the β-ray shielding assembly 2, and includes a display screen 9, an operation button 10, a dose measuring assembly, and an alerting or alarming assembly. The dose measuring assembly is used to measure the radiation dose received by the diseased site, and the display screen 9 is used to display the measured radiation dose received by the diseased site. When the dose or time to be received is about to reach, an alerting or alarming sound can be given by the alerting or alarming assembly, and the operation of the controller 1 can be controlled by the operation button 10, such as starting or stopping, and setting the dose alerting value.

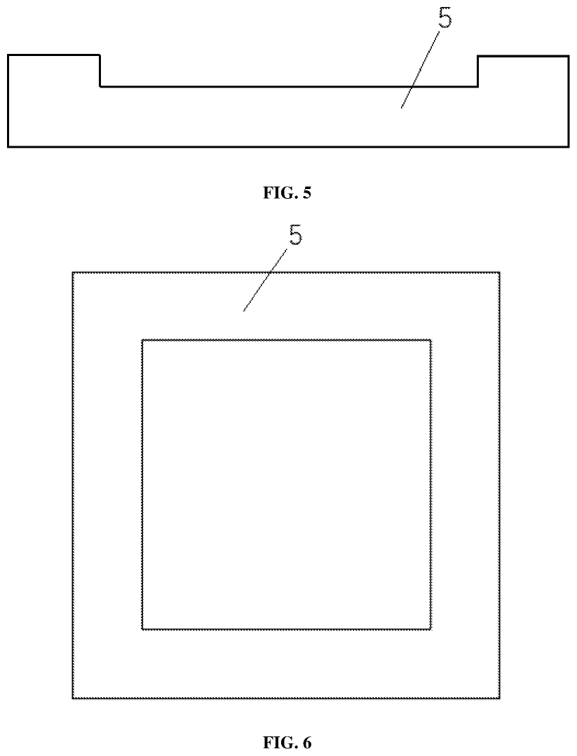

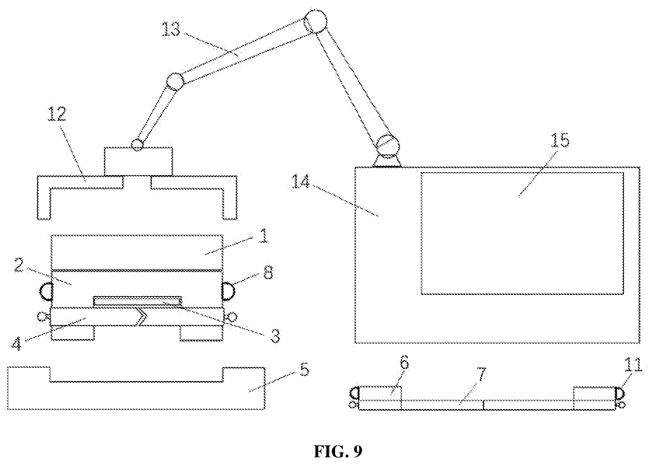

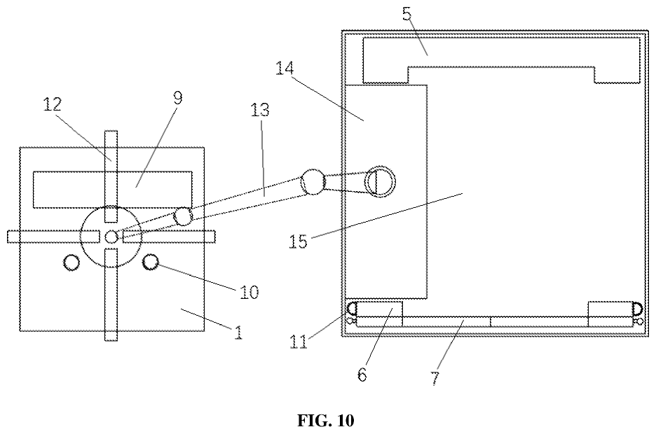

As shown in FIG. 5, FIG. 6, FIG. 9 and FIG. 10, the β-ray therapy apparatus includes an auxiliary device, including a supporting base 5, a host 14, and a mechanical arm 13. The supporting base 5 is provided with a groove for placing the therapy apparatus body. When the therapy apparatus is idle, the therapy apparatus body can be supported by the supporting base. The mechanical arm 13 can be fixedly mounted on the host 14 to support the movement of the mechanical arm 13. Meanwhile, the host 14 may also be provided with a closed storage area 15, which can be used for storing the supporting base 5 and the conformable assembly, as well as the therapy apparatus body. Both the supporting base 5 and the conformable assembly can be placed laterally in the closed storage area 15.

The mechanical arm 13 is provided with a clamp 12 for clamping the therapy apparatus body. Through the arrangement of the mechanical arm 13 and the clamp 12, on the one hand, the therapy apparatus body can be moved to the site to be treated by the mechanical arm 13, and kept at a corresponding position state for irradiation; on the other hand, the therapy apparatus body after use can be returned to the closed storage area 15.

When the apparatus provided by the present disclosure is used for treating hyperplastic diseases such as scar, hemangioma, tumor, circumscribed scleroderma, etc., β-ray irradiation windows with different opening sizes can be obtained by adjusting the positions of the blocking sheets 4, conformable areas with different opening sizes can be obtained by adjusting the conformable sheets 7, the application shape can be selected according to the shape, thickness and state of the lesion, and the irradiation range of the β-ray source 3 can be adjusted. The therapy apparatus body and the conformable assembly can be fixed to the surface of the human body through the elastic straps, and are not easy to fall off. The clamp 12 and the mechanical arm 13 can be used to fix a treatment position of the therapy apparatus and store the therapy apparatus into the closed storage area 15, thus facilitating use and recovery.

As shown in FIG. 1 to FIG. 10, the present disclosure further provides a method of using the β-ray therapy apparatus described above, including the following steps:

A conformable frame 6 is placed at a site to be treated, and can be fixed by second binding ring buckles 11 and elastic straps. Position of conformable sheets 7 are adjusted according to the shape of the site to be treated, thus forming a conformable area surrounding the site to be treated and achieving the effect of shape adjustment.

Ointment for burns, ointment for scars or gel is applied to the site to be treated, and uneven lesion is applied with the ointment or gel to the same level, making an area applied with the ointment or gel flush with the surfaces of conformable sheets 7, such that the irradiation dose on the site with thick lesion (thin applying thickness) is more, while the irradiation dose on the site with thin lesion (thick applying thickness) is less, thus different degrees of lesions can be irradiated with different intensities to achieve the effect of intensity modulation.

The therapy apparatus body can be buckled on the conformable frame 6, which can be clamped by the mechanical arm 13, or fixed by the first binding ring buckles 8 and the elastic straps. β-rays are emitted by the β-ray source 3, and the β-rays can irradiate the area applied with the ointment or gel through the conformable area.

In order to describe the method of using the present disclosure better, the following specific embodiments are provided.

If a patient has a scar to be treated on a forearm. The radiation dose needed for treatment is calculated according to the size of the scar and then is set as an alerting threshold of the controller 1. The shape of the β-ray irradiation window is adjusted to be the same as that of the scar by adjusting the position of the blocking sheet 4. The scar site is surrounded by the conformable assembly, then the conformable assembly is fixed by the elastic straps, and the conformable sheets 7 are adjusted to form a conformable area consistent with the shape of the scar. After a plane on the uneven scar site is formed by using the gel, the therapy apparatus body is fixed to the forearm of the patient through the elastic straps to start irradiation treatment. After reaching the irradiation dose required for treatment, the controller 1 gives an alerting sound, then the elastic straps can be loosened, and the therapy apparatus body is stored in the closed storage area 15 by the clamp 12 and the mechanical arm 13.

Specific examples are used herein for illustration of the principles and embodiments of the present disclosure. The description of the embodiments is merely used to help illustrate the method and its core principles of the present disclosure. In addition, a person of ordinary skill in the art can make various modifications in terms of specific embodiments and scope of application in accordance with the teachings of the present disclosure. In conclusion, the content of this specification shall not be construed as a limitation to the present disclosure.

1. A β-ray therapy apparatus, comprising a therapy apparatus body, and a conformable assembly, wherein the therapy apparatus body comprises a β-ray source, and a β-ray shielding assembly provided with a mounting chamber and a window communicating with the mounting chamber; the β-ray source is mounted in the mounting chamber; the conformable assembly comprises a conformable frame and a plurality of conformable sheets, a transmission area is formed in a middle of the conformable frame, the plurality of conformable sheets are arranged on the conformable frame, and are able to move towards or away from the transmission area; a conformable area surrounding a site to be treated is formed in the transmission area by adjusting positions of the plurality of conformable sheets; and the β-ray source emits β-rays towards the conformable area through the window.

2. The β-ray therapy apparatus according to claim 1, wherein the conformable frame is an annular frame, a hollow region of the annular frame is the transmission area, the plurality of conformable sheets are divided into two groups, and two groups of the plurality of conformable sheets are slidingly arranged on two opposite sides of the annular frame, respectively.

3. The β-ray therapy apparatus according to claim 1, wherein a periphery of the β-ray shielding assembly is provided with a plurality of first binding ring buckles, a periphery of the conformable frame is provided with a plurality of second binding ring buckles, and the first binding ring buckles and the second binding ring buckles are respectively configured for connecting with elastic straps.

4. The β-ray therapy apparatus according to claim 1, comprising a β-ray irradiation window, wherein the β-ray irradiation window is mounted at the window, and comprises one or more blocking sheets; and the blocking sheets move with respect to the window to change a size and shape of the window for adjusting an irradiation range of the β-rays emitted by the β-ray source.

5. The β-ray therapy apparatus according to claim 4, wherein the one or more blocking sheets are arranged on the β-ray shielding assembly in a penetrating manner, and move towards or away from the window to shield or open the window.

6. The β-ray therapy apparatus according to claim 5, wherein when a plurality of blocking sheets are provided, the plurality of blocking sheets are divided into two groups, and the two groups of blocking sheets are slidingly arranged on two opposite sides of the β-ray shielding assembly, respectively.

7. The β-ray therapy apparatus according to claim 6, wherein the two groups of blocking sheets are arranged in one-to-one correspondence to form a plurality of pairs of blocking sheets; in each pair of blocking sheets, one blocking sheet is provided with a V-shaped protrusion, and an other blocking sheet is provided with a V-shaped groove in fit with the V-shaped protrusion; and the V-shaped protrusion and the V-shaped groove extend in a width direction of the window.

8. The β-ray therapy apparatus according to claim 1, comprising a controller, wherein the controller is mounted on an outer side of the β-ray shielding assembly, and comprises a display screen, an operation button, a dose measuring assembly, and an alerting or alarming assembly.

9. The β-ray therapy apparatus according to claim 1, comprising an auxiliary device, wherein the auxiliary device comprises a supporting base, a host, and a mechanical arm; the supporting base is provided with a groove for placing the therapy apparatus body, the host is fixedly mounted on the mechanical arm, the host is provided with a closed storage area, and the mechanical arm is provided with a clamp for clamping the therapy apparatus body.

10. A method of using the β-ray therapy apparatus according to claim 1, comprising the following steps:
placing the conformable frame at the site to be treated, adjusting the positions of plurality of conformable sheets according to a shape of the site to be treated, thus forming the conformable area surrounding the site to be treated;
applying ointment or gel to the site to be treated, and making an area applied flush with surfaces of the plurality of conformable sheets; and
buckling the therapy apparatus body on the conformable frame, and emitting β-rays by the β-ray source, wherein the β-rays irradiate to the area applied through the conformable area.

11. The method according to claim 10, wherein the conformable frame is an annular frame, a hollow region of the annular frame is the transmission area, the plurality of conformable sheets are divided into two groups, and two groups of the plurality of conformable sheets are slidingly arranged on two opposite sides of the annular frame, respectively.

12. The method according to claim 10, wherein a periphery of the β-ray shielding assembly is provided with a plurality of first binding ring buckles, a periphery of the conformable frame is provided with a plurality of second binding ring buckles, and the first binding ring buckles and the second binding ring buckles are respectively configured for connecting with elastic straps.

13. The method according to claim 10, comprising a β-ray irradiation window, wherein the β-ray irradiation window is mounted at the window, and comprises one or more blocking sheets; and the blocking sheets move with respect to the window to change a size and shape of the window for adjusting an irradiation range of the β-rays emitted by the β-ray source.

14. The method according to claim 13, wherein the one or more blocking sheets are arranged on the β-ray shielding assembly in a penetrating manner, and move towards or away from the window to shield or open the window.

15. The method according to claim 14, wherein when a plurality of blocking sheets are provided, the plurality of blocking sheets are divided into two groups, and the two groups of blocking sheets are slidingly arranged on two opposite sides of the β-ray shielding assembly, respectively.

16. The method according to claim 15, wherein the two groups of blocking sheets are arranged in one-to-one correspondence to form a plurality of pairs of blocking sheets; in each pair of blocking sheets, one blocking sheet is provided with a V-shaped protrusion, and an other blocking sheet is provided with a V-shaped groove in fit with the V-shaped protrusion; and the V-shaped protrusion and the V-shaped groove extend in a width direction of the window.

17. The method according to claim 10, comprising a controller, wherein the controller is mounted on an outer side of the β-ray shielding assembly, and comprises a display screen, an operation button, a dose measuring assembly, and an alerting or alarming assembly.

18. The method according to claim 10, comprising an auxiliary device, wherein the auxiliary device comprises a supporting base, a host, and a mechanical arm; the supporting base is provided with a groove for placing the therapy apparatus body, the host is fixedly mounted on the mechanical arm, the host is provided with a closed storage area, and the mechanical arm is provided with a clamp for clamping the therapy apparatus body.

19. The β-ray therapy apparatus according to claim 2, comprising a β-ray irradiation window, wherein the β-ray irradiation window is mounted at the window, and comprises one or more blocking sheets; and the blocking sheets move with respect to the window to change a size and shape of the window for adjusting an irradiation range of the β-rays emitted by the β-ray source.

20. The β-ray therapy apparatus according to claim 3, comprising a β-ray irradiation window, wherein the β-ray irradiation window is mounted at the window, and comprises one or more blocking sheets; and the blocking sheets move with respect to the window to change a size and shape of the window for adjusting an irradiation range of the β-rays emitted by the β-ray source.
